(19) United States
(12) Patent Application Publication
(10) Pub. No.: US 2010/0191330 A1
(43) Pub. Date: Jul. 29, 2010

(54) HUMAN MAMMARY PROSTHETIC SUPPORT AND METHOD OF IMPLANTING

(76) Inventors: Gary Pierre Lauryssen, Gauteng (ZA); Hans DeBruijn, Hengelo (NL); Siegmund Johannes, Gauteng (ZA); Petrus Verster van Deventer, Cape Town (ZA)

Correspondence Address: MARSHALL, GERSTEIN & BORUN LLP, 233 SOUTH WACKER DRIVE, 6300 WILLIS TOWER, CHICAGO, IL, 60606-6357, US

(21) Appl. No.: 12/666,280

(22) PCT Filed: Jun. 24, 2008
(86) PCT No.: PCT/IB08/52503

§ 371 Date: Apr. 13, 2010

(30) Foreign Application Priority Data
Jun. 24, 2007 (ZA) 2007/00660

(51) Int. Cl. A61F 2/12

(52) U.S. Cl. 623/8

(57) ABSTRACT

A human mammary prosthetic support (5) is disclosed which comprises a continuous sheet of biocompatible mesh formed into the shape of a cup, securable between the skin and glandular structure of a human breast with an aperture (67) through which the nipple-areola structure is beatable. Also disclosed is a human mammary prosthetic support which comprises a continuous sheet of biocompatible mesh formed into a generally U-shaped formation, and methods to implant either of the supports into a human body.

### FIELD OF THE INVENTION

This invention relates to a mammary prosthetic support which is implantable into a human body and a method of implanting such a support in a human body.

### BACKGROUND TO THE INVENTION

For aesthetic reasons many women undergo breast surgery. This type of surgery includes purely aesthetic surgery such as breast enhancement surgery and reconstructive surgery necessitated through illnesses such as breast cancer. Irrespective of the reason for the surgery, it typically involves implanting a prosthesis in the form of a fluid filled bladder into one or both of the recipient's breasts to increase their size. The prosthesis is typically filled with saline or silicone gel to provide a texture as close to natural as possible.

Through very careful surgical technique it is possible, in some instances, to create an augmented or reconstructed breast which appears natural. However, in many instances it is impossible or at least very difficult to achieve the desired result and many recipients are left dissatisfied with their augmented or reconstructed breasts.

In many instances women do not wish to increase the size of their breasts but simply to reshape them to reverse, to some extent, the effects of ageing. In such cases implanting a prosthesis of the type described above will not necessarily produce the desired results.

There have been attempts to utilize prosthetic devices other than fluid filled bladders. These include various devices that are fixed in one way or another internally to the patient's body to internally support the breast tissue.

These devices have not proven to be successful. One of the reasons for their failure is the invasive techniques needed to secure them to the body, which include tapping screws into the sternum, clavicle, or ribs of a recipient. This requires the use of expensive screws and such procedures generally requires a substantial amount of time, which also increases the cost of the procedure.

Also relevant is the difficulty for surgeons to get predictable and repeatable results to ensure that a recipient receives what the surgeon had promised her, and that the breasts are post-operatively symmetrical.

Another problem with some of the devices is the techniques required to implant them. These produce excessive scarring and in some instances damage the structure of the nipple-areola structure. The latter may lead to loss of sensation in the nipple and/or areola which is a serious and undesirable side effect. This possible side effect may also prevent woman who still wish to breastfeed from making use of these procedures and devices.

### OBJECT OF THE INVENTION

It is an object of the invention to provide a human mammary prosthetic support and a method of implanting such a support which at least partly overcomes the abovementioned problems.

### SUMMARY OF THE INVENTION

In accordance with a first aspect of this invention there is provided a human mammary prosthetic support comprising a continuous sheet of biocompatible mesh formed into the shape of a cup, securable between the skin and glandular structure of a human breast with an aperture through which the nipple-areola structure is locatable.

There is further provided for the mesh to be resilient and for the cup to include a continuous curve between the aperture and the rim of the cup.

There is also provided for the cup to have a semi-ovoid shape, with a broad end and a small end.

There is further provided for the aperture to be located closer to the broad end than to the small end, and preferably for the distance between the aperture and the small end to be about the twice distance between the aperture and the broad end.

There is also provided for the rim of the cup to be reinforced by means of a rib extending around its circumference, and for the rim of the aperture to be reinforced by means of a rib extending around its circumference.

There is still further provided for the cup to have a predetermined size, preferably in the range of between 150 cc and 500 cc, more preferably one of a 350 cc, 400 cc, 450 cc or 500 cc size.

According to a further feature of the invention there is provided a human mammary prosthetic support kit comprising a support as described above and a complementary shaped and sized guide, preferably manufactured from a clear plastics material.

There is further provided for the guide to include at least one aperture, and preferably a plurality of apertures slots along the circumference of the guide, and for the apertures to be shaped and configured to allow a surgeon operatively to secure a support beneath the guide by securing means to tissue underlying the support.

There is still further provided for the apertures in the guide to comprise slots.

There is further provided for the underlying tissue to comprise the glandular structure of a breast and for the means to secure the support to the tissue surrounding the glandular structure, preferably the facia of the pectoral muscle underlying the glandular structure, to comprise stitches or staples made or inserted through the mesh of the support and the glandular structure.

There is still further provided for the kit to be sized in a plurality of predetermined sizes, preferably in sizes ranging from 150 cc to 500 cc, and more preferably in sizes 350 cc, 400 cc, 450 cc and 500 cc.

In accordance with a further feature of the invention there is provided a method of implanting a support of the type described above in a human body, the method including the steps of making at least one incision to gain access to the breast tissue of a recipient, degloving the skin and subcutaneous fascia from the glandular structure of the breast, inserting the support over the nipple-areola structure to locate the nipple-areola structure through the aperture, locating the small end of the support substantially adjacent the sternum of the recipient, spreading the support to contact the glandular structure over substantially the entire inner surface of the cup forming the support, locating a complimentary shaped guide over the support, securing the support to the tissue surrounding the glandular structure, preferably the facia of the pectoral muscle underlying the glandular structure, by attachment means through at least one aperture in the guide, removing the guide, securing the support, if required, by additional attachment means preferably along the rim of the support, and closing the incision or incisions in the breast.

There is further provided for the method to include the step of making a peri-areolar or “Wise”-incision to gain access to the breast tissue of a recipient.

According to a second aspect of this invention there is provided a human mammary prosthetic support comprising a continuous sheet of biocompatible mesh formed into a generally U-shaped formation with a base from which two projections extend, a first of the two projections operatively forming a medial arm and the second of the two projections operatively forming a lateral arm, the operatively outer edge of the base extending with a convex curve to the operatively outer edge of the medial arm, and the operatively outer edge of the base extending with a convex curve to a lateral point and from the lateral point with a concave curve to the operatively outer edge of the lateral arm, the support being securable between the skin and glandular structure of a human breast to form an aperture through which the nipple-areola structure is locatable.

There is further provided for the support to include a first support shaped and configured for a first type of breast and a second support shaped and configured for a second type of breast, for the outer edge of the base of the first support to include a convex curve which extends from the base to the lateral point to have a first length and for the concave curve which extends from the lateral point to the lateral arm to be directed substantially away from the medial arm; and for the operatively outer edge of the base of the second support to include a convex curve which extends from the base to the lateral point to have a second length which is greater than the first length of the first support, for the concave curve which extends from the lateral point to the lateral arm to be directed substantially towards the medial arm, and wherein the second type of breast includes substantially more breast tissue located laterally of the thorax of a woman compared to the first type of breast.

There is further provided for the inner edge of the lateral arm of the first support to have a convex shape which is directed substantially away from the medial arm.

There is also provided for the inner edge of the lateral arm of the second support to have a concave shape, alternatively a substantially straight edge, which extends substantially parallel with the inner edge of the medial arm from the base.

In accordance with a further feature of the invention there is provided a method of implanting a human mammary prosthetic support of the type described above in a human body, the method including the steps of making at least one incision to gain access to the breast tissue of a recipient, degloving the skin and subcutaneous fascia from the glandular structure of the breast, inserting the support with the medial arm located adjacent the sternum of the recipient, securing the end of the medial arm to the tissue above the breast tissue and underneath the clavicle proximate the sternum, extending the base and lateral arm of the support around the nipple-areola structure and over the glandular structure of the breast, overlaying and securing the end of the lateral arm at least partly over the end of the medial arm, preferably securing the outer edge of the support in at least one location to the glandular structure of the breast, and closing the at least one incision.

There is further provided for the method to include the step of shaping each breast individually by manipulating the extent and angle of overlay of the end of the lateral arm over the end of the medial arm.

There is also provided for the method to include the step of shaping the support to fit an individual breast by cutting the support, in particular cutting a cup formed between the medial arm and lateral arm above the base to accommodate the nipple and areola structure.

There is still further provided for the method to include enclosing breast tissue located laterally of the thorax of a recipient by securing the lateral point of the second support, as defined above, over such breast tissue to support it.

### BRIEF DESCRIPTION OF THE DRAWINGS

Preferred embodiments of the invention are described by way of example only and with reference to the accompanying drawings in which:

FIG. 1 is a front view of a human female with breast ptosis;

FIG. 2 is a side view of the female of FIG. 1;

FIG. 3 shows a perspective view of a first embodiment of a support used in the procedure;

FIG. 4 shows a perspective view of a guide complimentary to the support of FIG. 3;

FIG. 5 shows a “Wise”-incision made into a breast of the female of FIG. 1;

FIG. 6 shows a peri-areolar incision made into a breast of the female of FIG. 1;

FIG. 7 shows a set of guides of predetermined sizes being fitted to the exposed breast of the female of FIG. 1 to determine the correct size support for the breast;

FIG. 8 shows a series of boxes with supports from which a correctly sized support is taken based on the size of the guide of FIG. 7;

FIG. 9 shows a selected support being fitted to the exposed breast tissue;

FIG. 10 shows a selected guide being placed above the support;

FIG. 11 shows the support of FIG. 7 being secured to the breast tissue by means of stitches through small apertures in the template; shows the guide being removed after the support has been stitched to the breast tissue;

FIG. 13 shows the support being secured to the breast tissue by means of additional stitches;

FIG. 14 shows closure of the “Wise”-incision;

FIG. 15 shows closure of the peri-areolar incision;

FIG. 16 shows a front view of the female of FIG. 1 after the procedure;

FIG. 17 is a side view of the female of FIG. 1 after the procedure;

FIGS. 18A-D show a step-wise summary of the procedure showing two human female breasts, before and after the procedure, the incision and placement of the support during the procedure;

FIG. 19 shows a further embodiment of a guide according to the invention; and

FIG. 20 shows a support with a lateral extension of the rim complimentary to the guide of FIG. 19;

FIG. 21 is a plan view of a second embodiment of a support shaped for a first type of breast;

FIG. 22 is a front perspective view of the support of FIG. 21 in its implanted shape, showing only the support;

FIG. 23 is a side perspective view of the support of FIG. 22;

FIG. 24 is a plan view of a third embodiment of a support shaped for a second type of breast;

FIG. 25 is a front perspective view of the support of FIG. 24 in its implanted shape, showing only the support;

FIG. 26 is a side perspective view of the support of FIG. 25;

### DETAILED DESCRIPTION OF THE INVENTION

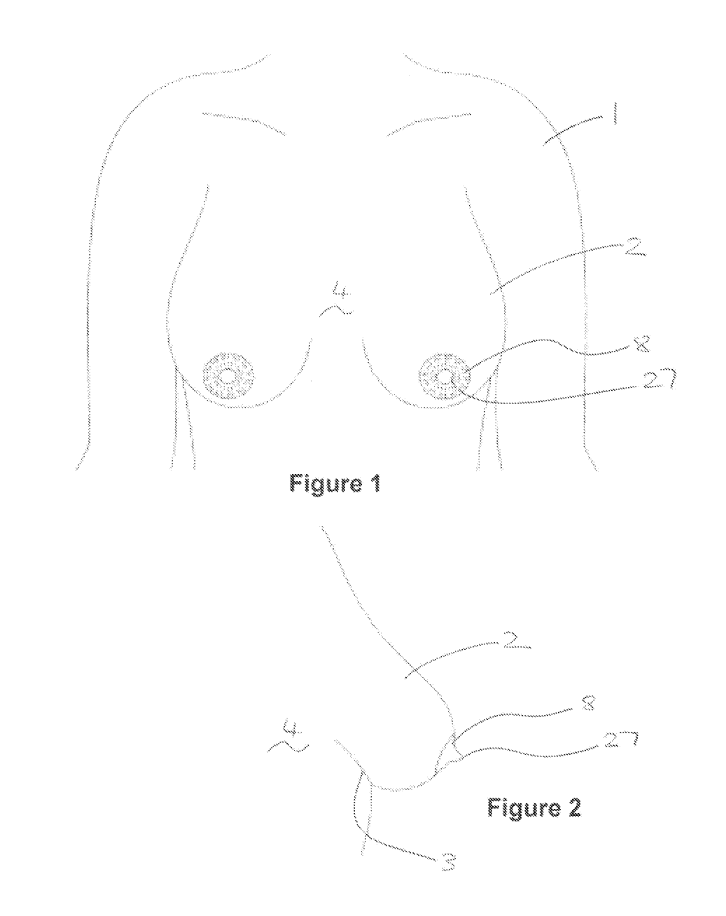

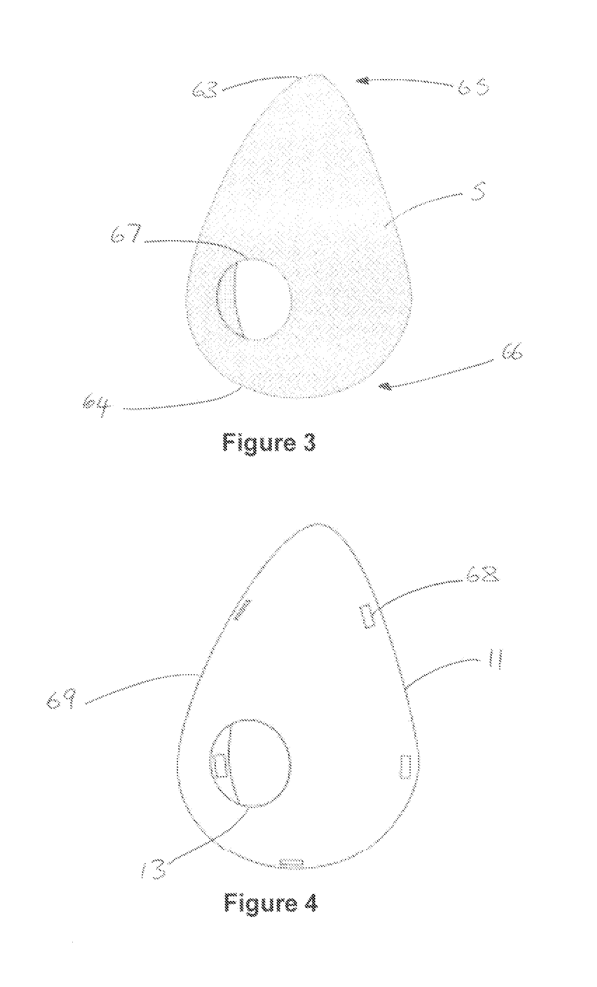

A first embodiment of a support (5), a guide (11) complimentary to the support (5), and a procedure to implant the support according to the invention is shown in FIGS. 3 to 15. FIGS. 1 and 2 show front and side views respectively of a female patient (1) with breast ptosis. FIG. 1 shows that the breasts (2) of the patient have drooped. This may be observed by observing the line of contact (3) between the skin of a breast (2) and the skin on the thorax (4) of the patient. As shown in FIG. 2, it is clear that the skin of the breast and the skin of thorax are in contact over a significant area, which is an indication of breast ptosis.

The support (5) is shown in FIG. 3. The support (5) comprises a mesh formed into a generally semi-ovoid shape. The support (5) has superior (63) and inferior (64) ends. The superior end (63) is formed by the small end (65) of the semi-ovoid shaped mesh and the inferior end (64) by the large end (66) of the semi-ovoid shaped mesh. An aperture (67) is formed centrally on a side-to side line through the mesh, and offset of centrally on an end-to-end line through the mesh located closer to the inferior end (64) than the superior end (63). The aperture (67) is shaped to be complimentary to an average size nipple-areola structure.

The guide (11) is manufactured from a clear resilient plastics material and is complimentarily shaped to the support (5). The guide (11) includes a series of elongate slots (68) spaced apart along its rim (69). The guide also includes an aperture (13) for the nipple-areola structure.

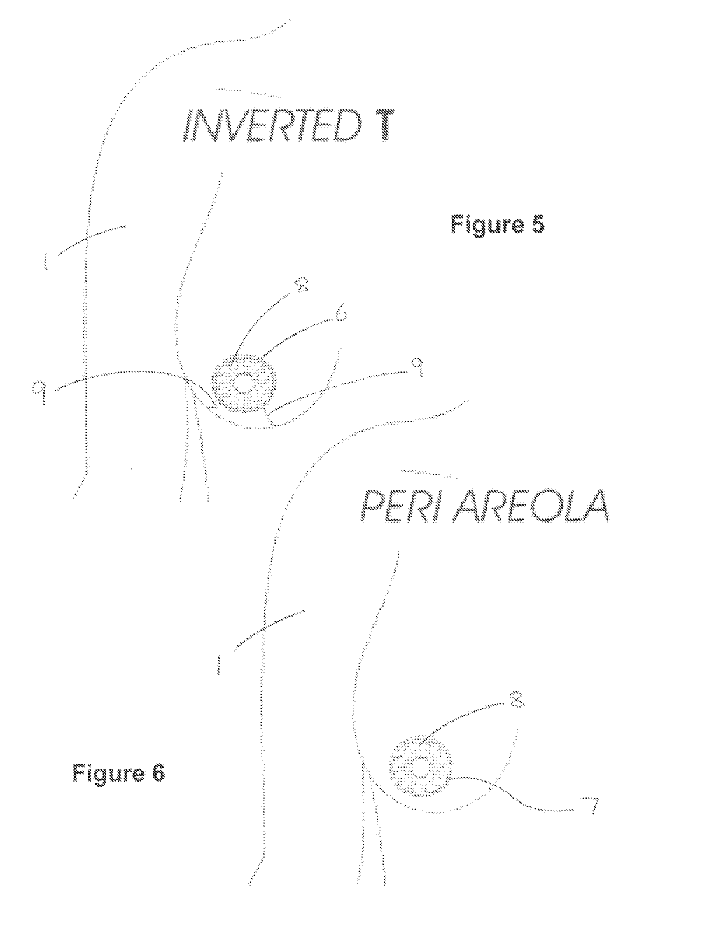

The procedure to implant the support (5) starts by making an incision into the breast (2). The incision may be a Wise-type or inverted T type incision (6) as shown in FIG. 5 or a peri-areolar incision (7) as shown in FIG. 6. The choice of incision depends on the surgeon and is chosen to best suit each specific patient. These incisions (6, 7) are well-established incisions used to gain access to the breast tissue during surgery.

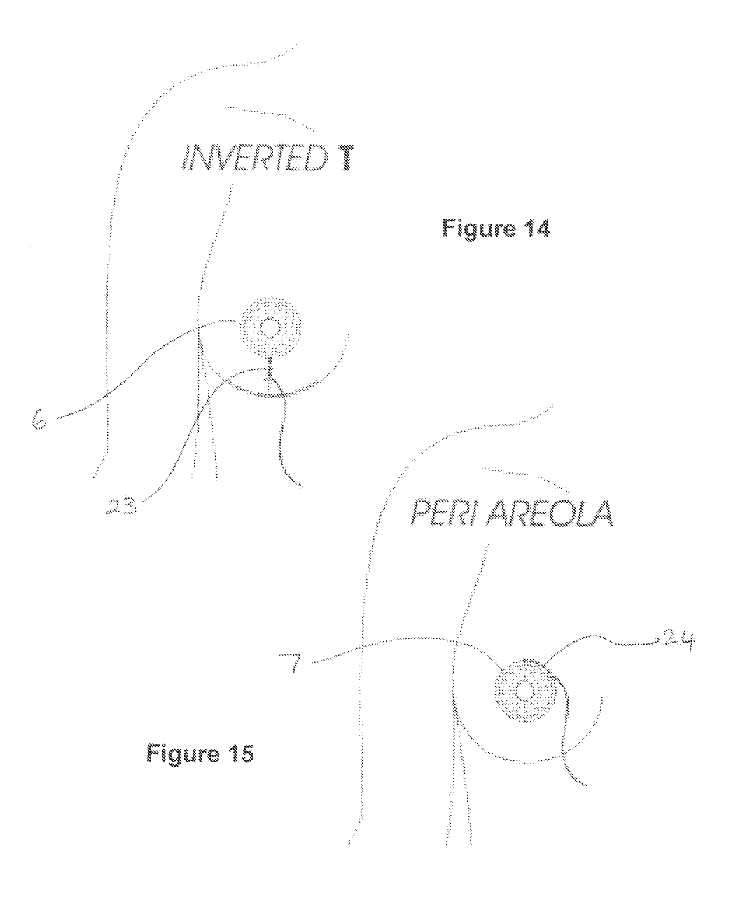

The Wise-type incision (6) comprises an incision which extends around the areola (8) as shown in FIG. 5 and downwards from the areola towards the line of interface (3) between the skin of the breast tissue and the skin of the thorax, from where the incision is extended to the sides in the form of an inverted T (9). As shown in FIG. 5, two of these incisions may be made side-by side, which in conjunction with the incision around the areola provides for maximum exposure of the breast tissue. Such an incision (9) is also used when an amount of skin needs to be removed where the size of the breast is reduced. The skin between the two incisions (9) is then removed. As shown further on in FIG. 14 a single line of suturing (28) is then used to join the two incisions (9).

The peri-areolar incision (7) comprises an incision around the areola (8). Once the incision has been made, the breast tissue is exposed by pulling the skin away from the areola (8). This type of incision (7) allows for access to the breast tissue with minimal scarring and without damaging the areolar structure.

Once the incision (6, 7) has been made the surgeon proceeds to deglove the skin and subcutaneous facia from the glandular structure of the breast, which exposes the glandular structure (10) to the surgeon. In particular, the superior-areola incision is carried into the subcutaneous tissue and a dissection performed in the subcutaneous plane superior to the subclavicular, sterno-clavicular and anterior axillary regions and medially to the parasternal region as well as laterally to the anterior axillary line, around the breast with adequate flap thickness.

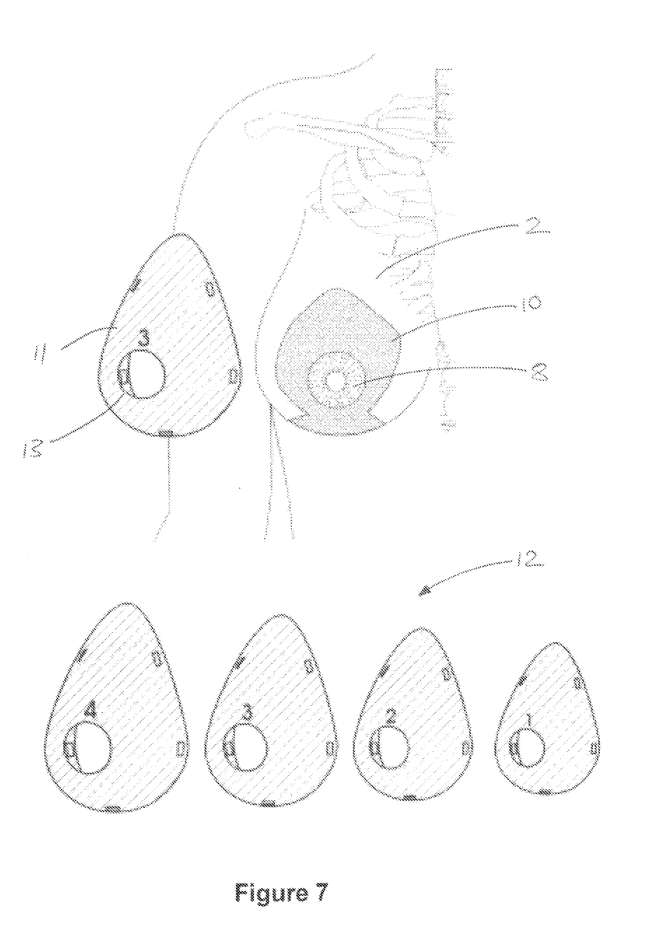

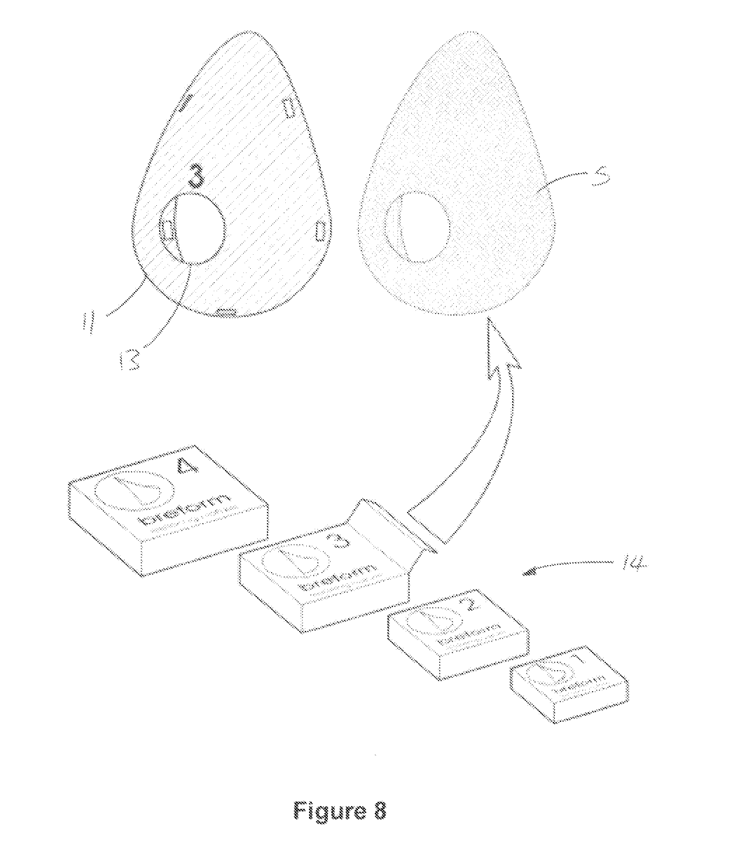

As shown in FIG. 7, a preformed guide (11) which is complementary shaped to the support (5) is used as a sizer to determine the correct size of support to use on the breast (2). A series of incrementally differently sized prostheses (12) and re-usable guides is used from which the correct size will be determined. The correct size support is determined by inserting the sterile guide (11) into the exposed breast between the glandular structure and the skin. The guide (11) includes an aperture (13) for the areola and nipple structure. By locating the guide (11) over the exposed glandular structure (10) in this manner, the surgeon is able to determine which guide has the correct size to hold the breast correctly. If a too large guide is chosen, there will not be perfect fit between the guide (11) and the glandular structure (10) which will be visible through the clear guide (11). If a too small guide (11) is used, it will not be possible to locate all of the breast tissue underneath it. Once the correct size guide (11) has been determined, it is possible to select the correct sized support (5) which is marked accordingly. The support (5) is then obtained from its sterile and sealed container (14), as shown in FIG. 8.

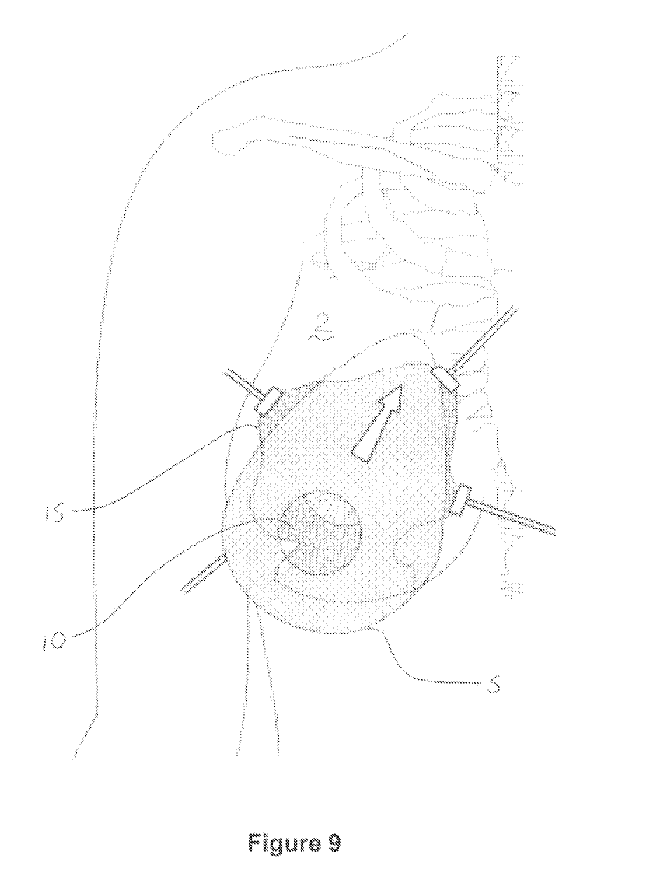

As shown in FIG. 9, the next step is to insert the correctly sized support (5) into the breast (2). The support (5) is inserted between the skin (15) and the exposed glandular structure (10) by pulling the skin (15) away and inserting the support by hand into the exposed area. The skin (15) is pulled away to allow access to the extent needed for this step.

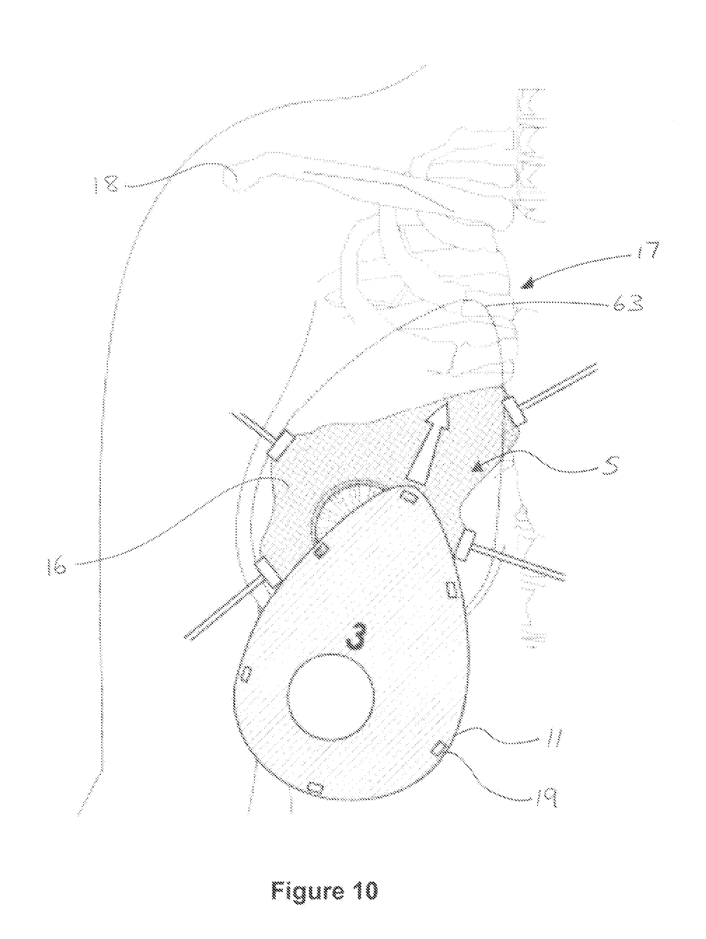

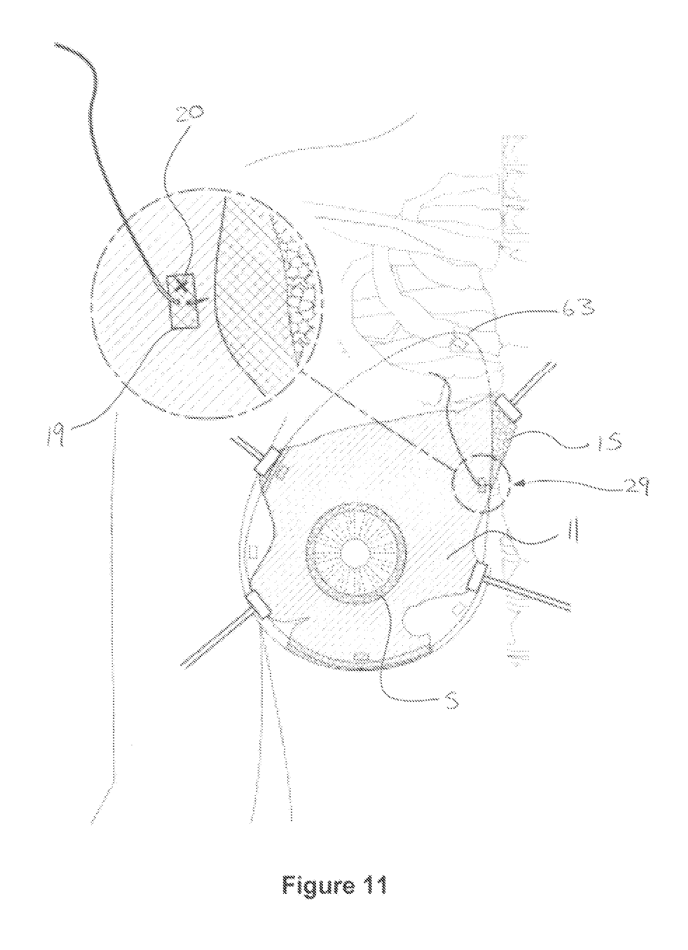

As shown in FIG. 10, the support is inserted such that its superior end (63) extends to about the 2^{nd} sterno costal junction (17), which is above the breast tissue and below the clavicle (18). To locate, smooth out, and secure the support, the correctly sized guide (11) is located over the support (5). The guide (11) is used to smooth out the mesh (16) from which the support is manufactured. Once the mesh (16) has been smoothed out and located correctly, the support (5) is secured in position by means of a number of locating stitches (20) which are made through a series of circumferential slots (19) in the guide (11) to the tissue surrounding the glandular structure, preferably the facia (29) of the pectoral muscle underlying the glandular structure. This is shown in FIG. 11. Once the support (5) has been secured in this manner to the underlying facia (29) of the pectoral muscle, the guide (11) is removed, as is shown in FIG. 12.

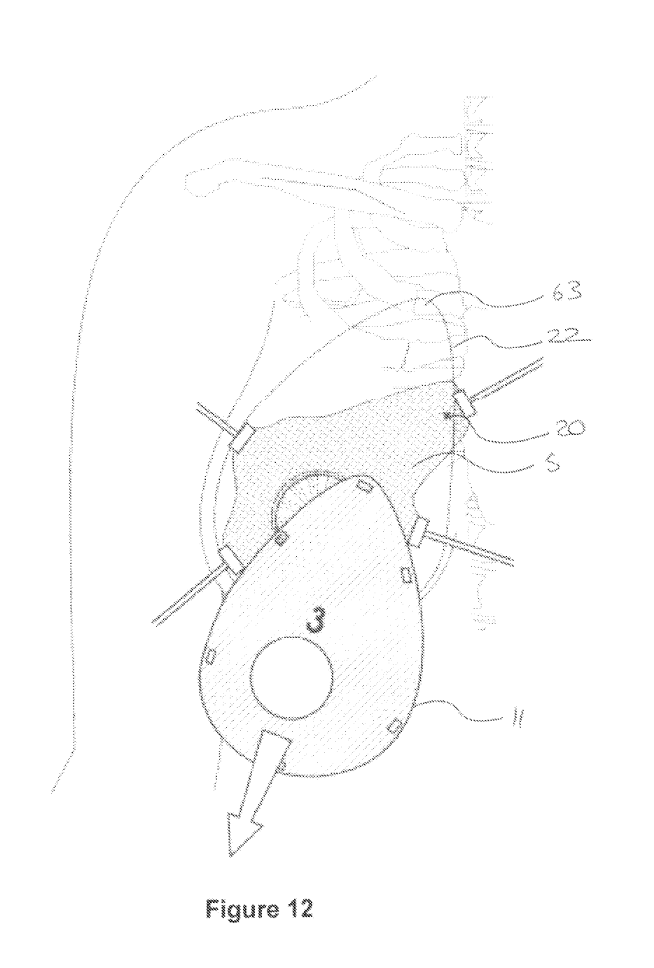

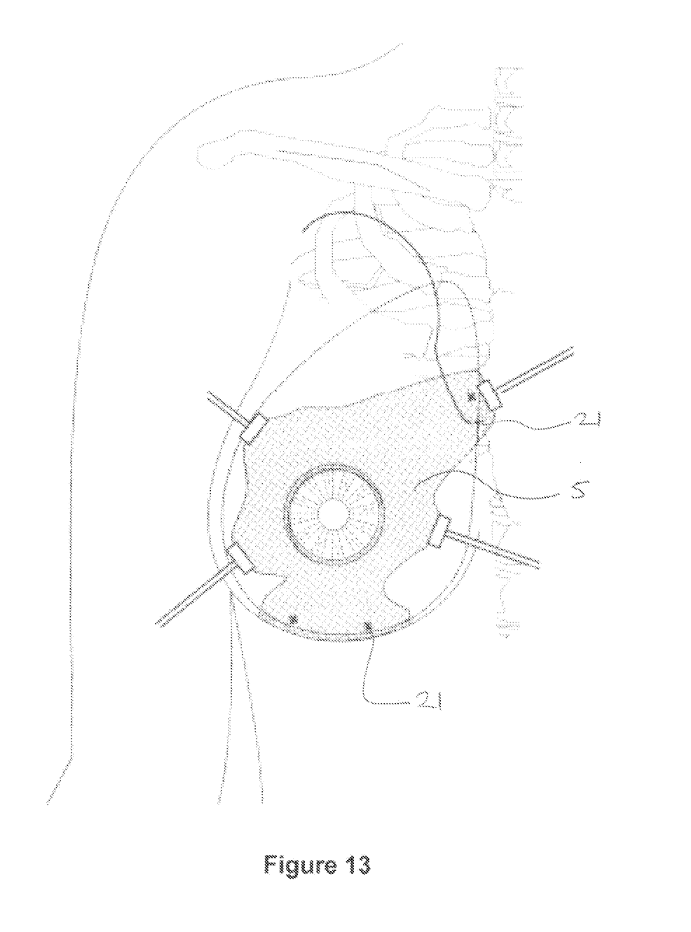

With the guide (11) removed further stitches (21) are used to secure the support to the tissue surrounding the glandular structure, preferably the facia of the pectoral muscle underlying the glandular structure, as shown in FIG. 13. These stitches (21) are spaced apart, preferably at regular intervals, along the rim (22) of the support (5) to secure it to the tissue surrounding the glandular structure (10).

After the support (5) has been secured in this manner, the breast (2) can be closed by suturing (23, 24) the incisions closed, as shown in FIGS. 14 and 15 respectively for the two types of incisions (6, 7) mentioned.

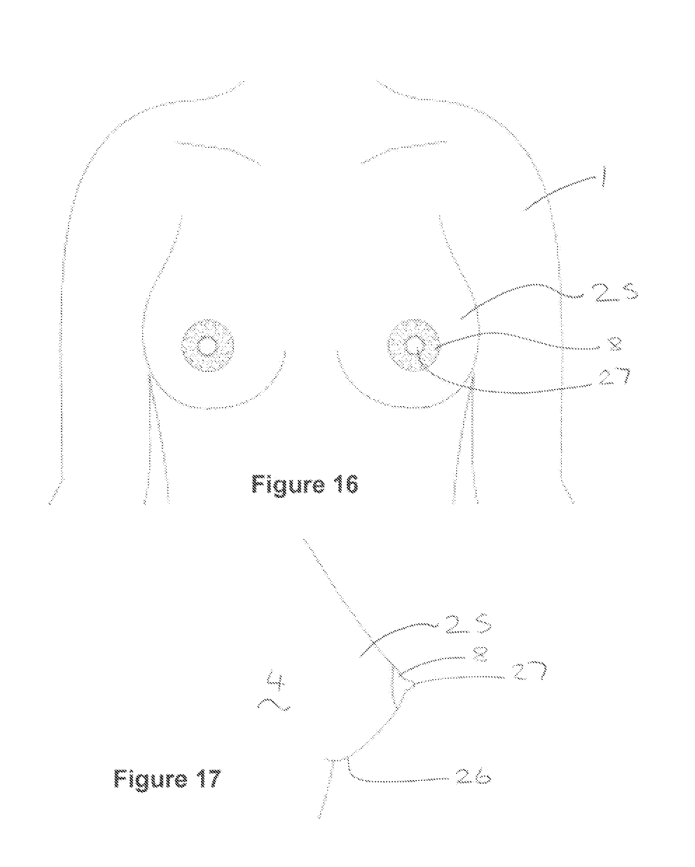

With the support in place and the incisions (6, 7) closed up, the support (5) serves to support the breast (2) in position. The supported breasts (25) are shown in FIGS. 16 and 17 in which the effects of implanting the prostheses (5) can be seen. By comparing FIGS. 2 and 17 it can be seen that the degree of contact (3, 26) between the skin of the breast and the skin of the thorax (4) is much less after the procedure. Another notable improvement may be appreciated by comparing the position and orientation of the areolas (8) and nipples (27) between FIGS. 1 and 16, and between FIGS. 2 and 17. After the procedure, as shown in FIG. 17, the areolas (8) are located higher on the thorax (4) as shown in FIG. 16 as opposed to the lower position of the areolas (8) shown in FIG. 2. After the procedure the nipples (27) are pointing to just above the horizontal as shown in FIG. 17 as opposed to pointing downwards as shown in FIG. 2.

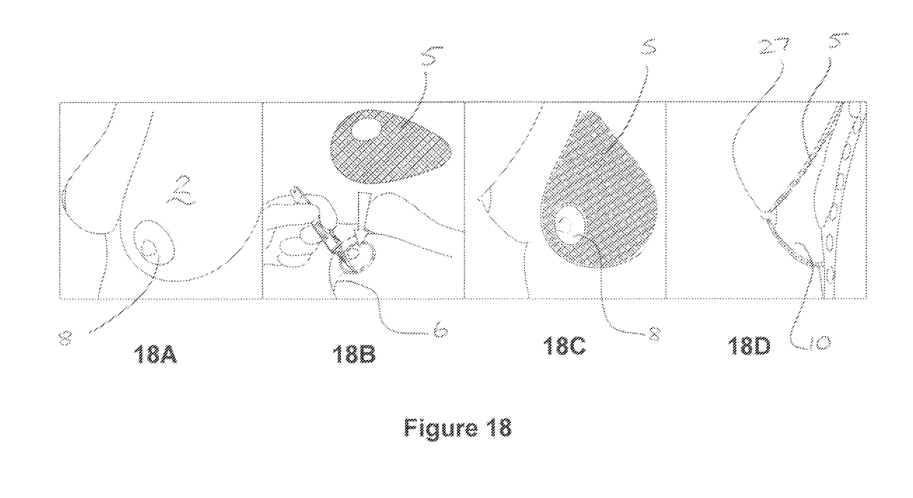

The entire procedure can be summarised by the drawings shown in FIGS. 18A to 18D. The breasts (2) of a recipient are shown in FIG. 18A before the procedure. Each breast (2) is then incised (7) to insert a support (5) according to the invention underneath the skin (15), as shown in FIG. 18B. After the procedure the support (5) supports the breast similar to how a brassiere would support the breast tissue but doing so from underneath the skin, as shown in FIG. 18C. This is also apparent when viewing the breast in cross section, as seen in FIG. 18D, which shows the support (5) supporting the breast tissue (10).

The surgical method to implant the support therefore includes the steps of:
i) making a peri-areolar or “Wise”-incision to gain access to the breast tissue of a recipient;
ii) degloving the skin and subcutaneous fascia from the glandular structure of the breast;
iii) inserting the support over the nipple and areola structure for the areola and nipple structure to extend through the aperture;
iv) locating the superior end of the support substantially adjacent the sternum of the recipient;
v) spreading the support to contact the glandular structure over substantially the entire inner surface of the wall of the support;
vi) locating a complimentary shaped guide over the support;
vii) securing the support to the tissue surrounding the glandular structure, preferably the facia of the pectoral muscle underlying the glandular structure, by attachment means through at least one slot in the guide;
viii) removing the guide and securing the support, if required, by additional attachment means preferably along the rim of the support; and
ix) and closing the incision or incisions in the breast.

A “Wise”-incision looks like an upside-down T, or anchor, and leaves a scar that circles around the areola and extends below the breast until it joins a horizontal scar—the top of the inverted T or curve of the anchor—below the breast.

A “peri-areolar” incision circles the areola on the edge of the areola, which leaves less visible scarring.

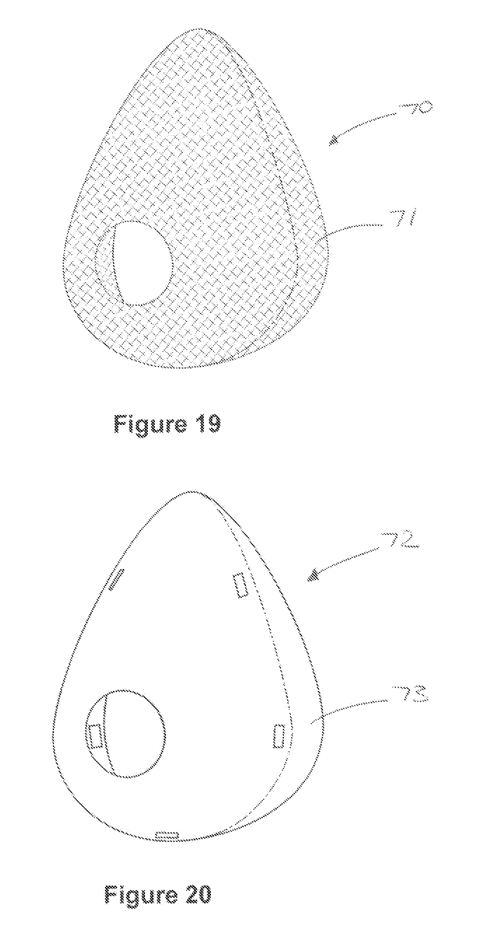

A second embodiment of a support (70) according to the invention is shown in FIG. 19, and a guide (72) complimentary to it in FIG. 20. This support (70) is similar to the support (5) according to the first aspect of the invention, apart from the fact that it includes a lateral extension (71) of the rim. The guide (72) also includes a complimentary extension (73) from its rim. The aim of this extension (71) is to support breast tissue that is located at the side of the thorax of a recipient, as is often the case with recipients with large breasts. This is similar to the larger support (50) of the two prostheses (31, 50) according to the second aspect of the invention. In such a case the support (70) can still be used for the left or right breast simply by inverting the support (70), since it is not outside or inside specific.

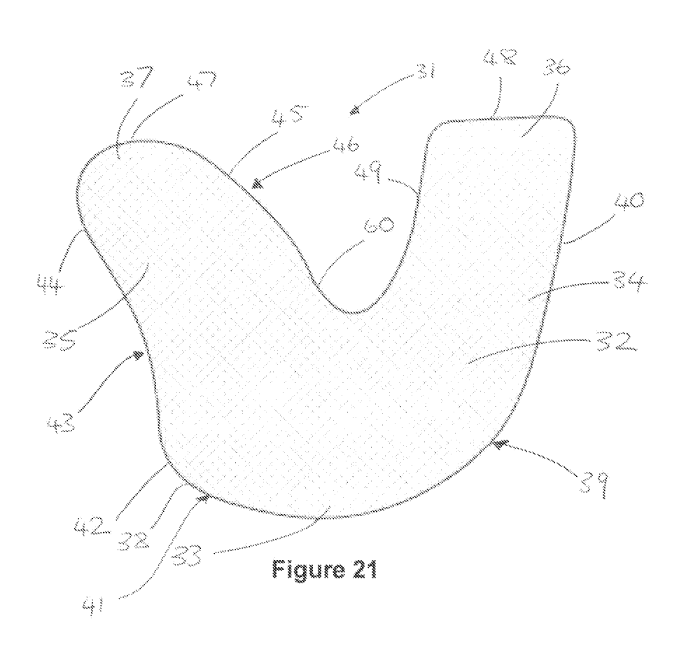

Third and fourth embodiments of supports (31, 50) according to invention are shown in FIGS. 21 to 26. The support (31) in FIG. 21 comprises a biocompatible resilient mesh (32) shaped in a generally U-shaped formation with a base (33) from which two projections (34, 35) extend. A first (34) of the two projections forms, after it has been implanted, a medial arm (36) and the second (35) of the two projections similarly forms a lateral arm (37) of the support.

The outer edge (38) of the base (33) extends with a convex curve (39) to the outer edge (40) of the medial arm (36). The outer edge (38) of the base (33) also extends with a convex curve (41) to a lateral point (42) and from the lateral point (42) with a concave curve (43) to the outer edge (44) of the lateral arm (37).

The concave curve (43) extends substantially away from the medial arm (36), which as can be seen from FIG. 21 directs the lateral arm (37) away from the medial arm (36). The inner edge (45) of the lateral arm (37) has a convex curve (46) and this is similarly curved substantially away from the medial arm (36).

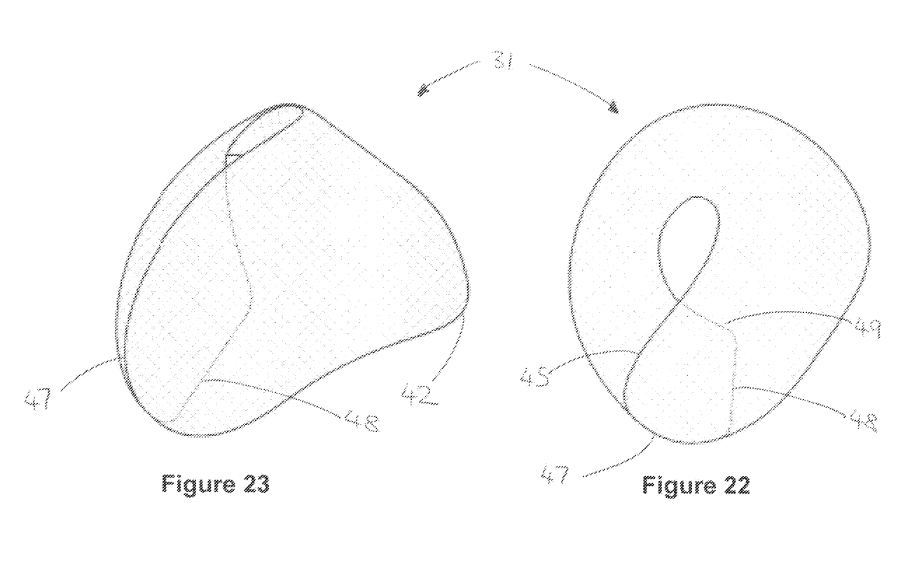

As shown in FIGS. 22 and 23, the support (31) in its implanted condition has the end (47) of the lateral arm (37) overlaying and secured to the end (48) of the medial arm (36) and the underlying tissue (not shown). The method of implanting the support (31) and securing the ends (47, 48) is described in more detail below.

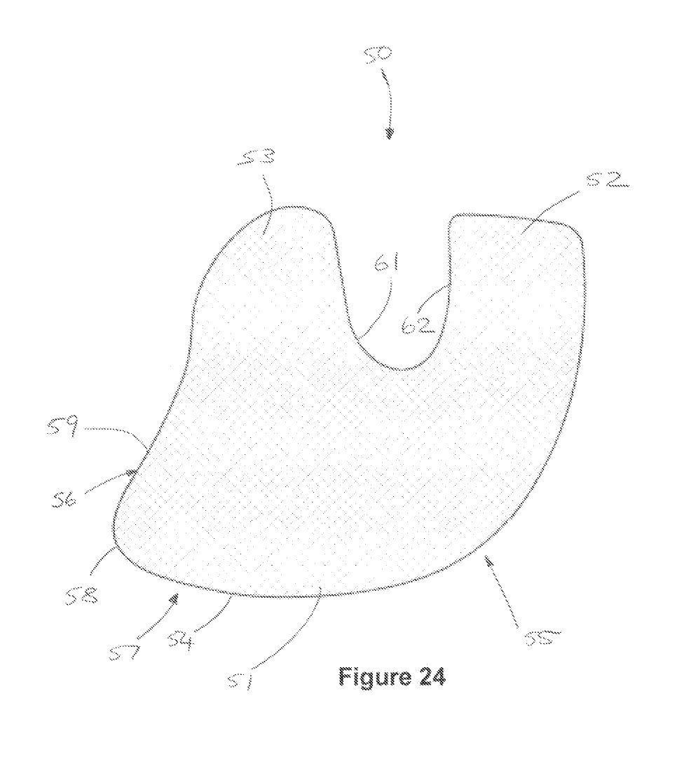

This support (31) is suitable for a recipient who has what may be described as normal to medium sized breasts and, importantly, who does not have any substantial amount of breast tissue located to the side of her thorax, i.e. almost under her arm in her armpit. In instances where this is the case, as is often found with a woman with larger breasts, the support (31) shown in FIGS. 21 to 23 may not be adequate to support the breast tissue located at the side of her thorax. In such a case a support (50) as shown in FIGS. 24 to 26 is more desirable.

The outer edge (54) of the base (51) of the support (50) shown in FIG. 24 has a convex curve (55) which extends from the base (51) to the medial arm (52), similar to that of the support (31) shown in FIGS. 21 to 23. The outer edge (54) of the base (51) of the support (50) also extends with a convex curve (57) to the lateral point (58). However, as is notable from a comparison of FIGS. 21 and 24, the length of this convex curve (57) is substantially longer in the support (50) of FIG. 24 than in the support (31) of FIG. 21.

The effect of this is that the lateral point (58) is located further away from the centre of the base (51), essentially to the side of the support (50), compared with the support (31) of FIG. 21. The convex curve (56) of the outer edge (59) which leads from the lateral point (58) to the lateral arm (53) is curved towards the medial arm (52), as opposed to away from the medial arm (36) in the support (31) of FIGS. 21.

Another notable difference is at the inner edge (60) of the lateral arm (37), which in the case of the support (31) of FIGS. 21 to 23 has a convex shape which extends substantially away from the inner edge (49) of the medial arm (36). In the support (50) of FIGS. 24 to 26 the inner edge (61) of the lateral arm (53) has a concave shape, which extends substantially parallel with the inner edge (62) of the medial arm (52) away from the base (51).

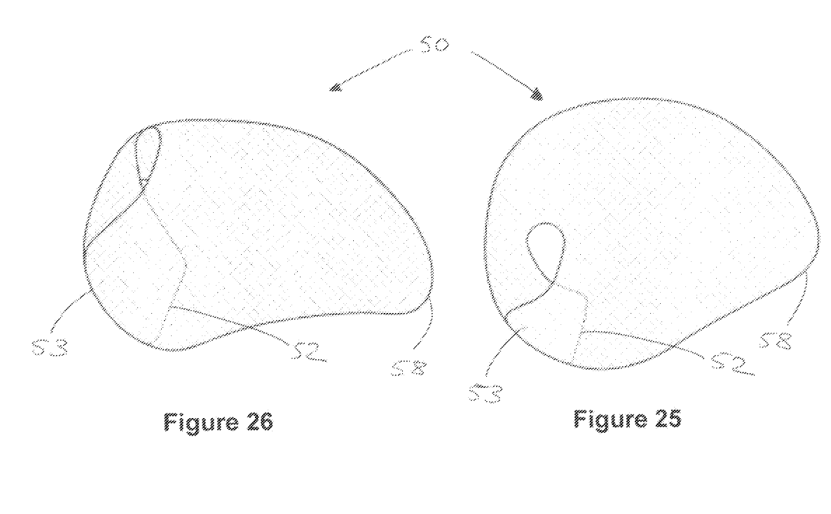

The effect of the difference in shapes is clear on viewing FIGS. 21 and 24. As mentioned above the support (50) of FIG. 24 has a lateral point (58) which projects substantially more to the side of the support (50) compared to the lateral point (42) of the support (31) of FIG. 21. It is this lateral point (58) of the support (50) of FIG. 24 which is used to support breast tissue that are located at the side of the thorax of a recipient, as is often the case with recipients with large breasts. The effect may be appreciated by comparing FIGS. 22 and 23 with FIGS. 25 and 26. The lateral point (58) of the support (50) shown in FIGS. 25 and 26 is securable to the side wall of the thorax, extending underneath the arm in the direction of the armpit of the recipient, to support breast tissue located there.

The two prostheses (31, 50) shown in FIGS. 21 to 26 support the breast tissue by effectively extending below the breast tissue (viewed from the point of view of a recipient standing upright), and shape the breast tissue to a desired shape. The adjustability of each individual support (31, 50), by movement of the amount and angle of overlap of the lateral arm (37, 53) over the medial arm (36, 52) of the support (31, 50) before securing the lateral arm (37, 53) in position, allows a surgeon to skillfully shape the two breasts of a recipient to be symmetrical to each other. The resilience of the mesh (32) from which the support (31, 50) is manufactured allows for natural expansion of a breast supported by the support. The nature of the mesh (32) and the procedure used to implant the support (31, 50) also allows the support (31, 50) to be cut during the implanting thereof, should it be required.

The surgical method to implant the support includes the steps of
i) making a peri-areolar or “Wise”-incision to gain access to the breast tissue of a recipient;
ii) degloving the skin and subcutaneous facia from the glandular structure of the breast;
iii) inserting the support with the medial arm located adjacent the sternum of the recipient; iv) securing the top end of the medial arm to the 2nd sterno-costal junction;
v) extending the base and lateral arm of the support around the nipple areolar complex to support the glandular structure, attaching it all the way to the chest wall;
vi) overlaying and securing the end of the lateral arm at least partly over the medial arm, preferably securing the outer edge of the support in at least one location to the glandular structure of the breast;
vii) and closing the incision or incisions.

The method of implanting the support also includes shaping each breast by manipulating the amount and angle of overlay of the end of the lateral arm over the end of the medial arm. This allows the surgeon to obtain symmetry between the two breasts of a recipient, to a degree that is difficult if not impossible to achieve with conventional mammary prosthetic devices.

The method also includes shaping the support to fit an individual breast by cutting the support, in particular cutting the cup formed between the medial and lateral arms above the base to accommodate the nipple structure.

In the case of a recipient with large breasts for which the support shown in FIGS. 24 to 26 will be used the breast tissue located laterally to the thorax, i.e. in the vicinity of the armpit of the recipient, is supported and shaped by securing the lateral point of the support over such breast tissue. This minimizes, at least to some extent, the natural tendency of large breasts from sagging to the side of a woman's thorax when she lies on her back.

It will be appreciated that the embodiments described above are given by way of example only and changes to the embodiments are possible without departing from the scope of the invention.

It is also possible to secure the support (5, 31, 50, 70) by means other than stitching, for example by means of staples.

The use of the guide will allow a surgeon to assess asymmetry between two breasts by noting the size guide that fits each breast best. This will allow the surgeon to reduce the size of the larger of two adjacent breasts which will in turn improve symmetry between them.

1. A human mammary prosthetic support comprising a continuous sheet of biocompatible mesh formed into the shape of a cup, securable between the skin and glandular structure of a human breast with an aperture through which the nipple-areola structure is locatable.

2. A support as claimed in claim 1 in which the mesh is resilient.

3. A support as claimed in claim 1 in which the cup includes a continuous curve between the aperture and the rim of the cup.

4. A support as claimed in claim 1 in which the cup has a semi-ovoid shape, with a broad end and a small end.

5. A support as claimed in claim 4 in which the aperture is located close to the broad end that to the small end.

6. A support as claimed in claim 4 in which the distance between the aperture and the small end is about the twice distance between the aperture and the broad end.

7. A support as claimed in claim 1 in which the rim of the cup is reinforced by means of a rib extending around its circumference.

8. A support as claimed in claim 1 in which the rim of the aperture is reinforced by means of a rib extending around its circumference.

9. A support as claimed in claim 1 which the cup has a predetermined size.

10. A support as claimed in claim 9 in which the size of the cup is in the range of between 150 cc and 500 cc.

11. A support as claimed in claim 9 in which the size of the cup has a size of 350 cc, 400 cc, 450 cc or 500 cc.

12. A human mammary prosthetic support kit comprising a support as claimed in claim 1 and a guide complementary shaped and sized to the support.

13. A kit as claimed in claim 12 in which the guide is manufactured from a layer of clear plastics material.

14. A kit as claimed in claim 12 in which the guide includes at least one aperture proximate the rim of the guide and the aperture is shaped and configured to allow a surgeon to secure the support beneath the guide, by securing means, to tissue underlying the support.

15. A kit as claimed in claim 12 in which the guide includes a plurality of apertures arranged along the circumference of the guide.

16. A kit as claimed in claim 15 in which the apertures are equidistantly spaced apart.

17. A kit as claimed in claim 14 in which the, or each, aperture is shaped in the form of a slot.

18. A kit as claimed in claim 12 in which the kit is sized in a plurality of predetermined sizes.

19. A kit as claimed in claim 18 in which the kit is sized in sizes ranging from 1.50 cc to 500 CC.

20. A kit as claimed in claim 18 in which the kit is sized in any one or more of sizes 350 cc, 400 cc, 450 cc and 500 cc.

21. A method of implanting a human mammary prosthetic support, as claimed in claim 1, in a human body, the method including the steps of making at least one incision to gain access to the breast tissue of a recipient, degloving the skin and subcutaneous fascia from the glandular structure of the breast, inserting the support over the nipple and areola structure to locate the areola and nipple structure through the aperture, locating the small end of the support substantially adjacent the sternum of the recipient, spreading the support to contact the glandular structure over substantially the entire inner surface of the cup forming the support, locating a guide as claimed in any one of claims 12 to 20 over the support, securing the support to the tissue surrounding the glandular structure by attachment means through at least one aperture in the guide, removing the guide, if required securing the support by additional attachment means to the tissue surrounding the glandular structure, and closing the incision or incisions in the breast.

22. A method as claimed in claim 21 in which the support is secured to the facia of the pectoral muscle underlying the glandular structure.

23. A method as claimed in claim 21 in which the support is secured along its rim to the tissue surrounding the glandular structure.

24. A method as claimed in claim 21 which includes making a peri-areolar or “Wise”-incision to gain access to the breast tissue of a recipient.

25. A human mammary prosthetic support comprising a continuous sheet of biocompatible mesh formed into a generally U-shaped formation with a base from which two projections extend, a first of the two projections operatively forming a medial arm and the second of the two projections operatively forming a lateral arm, the operatively outer edge of the base extending with a convex curve to the operatively outer edge of the medial arm, and the operatively outer edge of the base extending with a convex curve to a lateral point and from the lateral point with a concave curve to the operatively outer edge of the lateral arm, the support being securable between the skin and glandular structure of a human breast to form an aperture through which the nipple-areola structure is locatable.

26. A support as claimed in claim 25 which is shaped and configured for a first type of breast, in which the operatively outer edge of the base with the convex curve that extends from the base to the lateral point has a first length, and the concave curve that extends from the lateral point to the lateral arm extends substantially away from the medial arm.

27. A support as claimed in claim 26 in which the inner edge of the lateral arm of the first support has a convex shape which extends substantially away from the medial arm.

28. A support as claimed in claim 25 which is shaped and configured for a second type of breast which comprises a greater volume than a first type of breast, in which the operatively outer edge of the base of with the convex curve that extends from the base to the lateral point has a second length which is greater than the first length of the first support, and the concave curve that extends from the lateral point to the lateral arm extends substantially towards the medial arm.

29. A support as claimed in claim 28 in which the inner edge of the lateral arm of the second support has a concave shape, alternatively a substantially straight edge, which extends substantially parallel with the inner edge of the medial arm from the base.

30. A method of implanting a support as claimed in claim 25, including the steps of making at least one incision to gain access to the breast tissue of a recipient, degloving the skin and subcutaneous fascia from the glandular structure of the breast, inserting the support with the medial arm located substantially adjacent the sternum of the recipient, securing the end of the medial arm to the tissue above the breast tissue and underneath the clavicle proximate the sternum, extending the base and lateral arm of the support around the nipple and areola structure and over the glandular structure of the breast, overlaying and securing the end of the lateral arm at least partly over the end of the medial arm, and closing the at least one incision.

31. A method as claimed in claim 30 which includes, before the step of closing the at least one incision, the step of securing the outer edge of the support in at least one location to breast tissue.

32. A method as claimed in claim 30 which includes shaping a breast by manipulating the extent and angle of overlay of the end of the lateral arm over the end of the medial arm.

33. A method as claimed in claim 30 shaping the support to fit an individual breast by cutting the support.

34. A method as claimed in claim 33 in which the cup formed between the medial arm and lateral arm above the base to accommodate the nipple and areola structure is cut to fit an individual breast.

35. A method as claimed in claim 30 which includes enclosing breast tissue located laterally of the thorax of a recipient by securing the lateral point of the second support comprising a continuous sheet of biocompatible mesh formed into a generally U-shaped formation with a base from which two projections extend, a first of the two projections operatively forming a medial arm and the second of the two projections operatively forming a lateral arm, the operatively outer edge of the base extending with a convex curve to the operatively outer edge of the medial arm, and the operatively outer edge of the base extending with a convex curve to a lateral point and from the lateral point with a concave curve to the operatively outer edge of the lateral arm, the support being securable between the skin and glandular structure of a human breast to form an aperture through which the nipple-areola structure is locatable, which is further shaped and configured for a second type of breast which comprises a greater volume than a first type of breast, in which the operatively outer edge of the base of with the convex curve that extends from the base to the lateral point has a second length which is greater than the first length of the first support, and the concave curve that extends from the lateral point to the lateral arm extends substantially towards the medial arm over such breast tissue support such breast tissue.
